(19) United States
(12) Patent Application Publication
(10) Pub. No.: US 2016/0374918 A1
(43) Pub. Date: Dec. 29, 2016

(54) ENCAPSULATED SKIN CARE AGENT

(71) Applicant: The Procter & Gamble Company, Cincinnati, OH (US)

(72) Inventors: Jiten Odhavji DIHORA, Liberty Township, OH (US); Taotao ZHU, West Chester, OH (US); Todd Laurence UNDERINER, Cincinnati, OH (US); Jesus VELAZQUEZ, West Chester, OH (US)

(21) Appl. No.: 15/196,924

(22) Filed: Jun. 29, 2016

Related U.S. Application Data
(60) Provisional application No. 62/186,012, filed on Jun. 29, 2015.

(51) Int. Cl. A61K 8/60; A61K 8/11; A61Q 19/08; A61K 8/02; A61K 8/34

(52) U.S. Cl. CPC A61K 8/602; A61K 8/0241; A61K 8/345; A61Q 19/08; A61K 8/11; A61K 2800/412; A61K 2800/413; A61K 2800/52

(57) ABSTRACT

A stable cosmetic agent that includes an encapsulated particle in a core-shell configuration. The encapsulated particle is formed by coating nicotinamide riboside powder or a nicotinamide riboside containing material with one or more encapsulating agents. The stable encapsulated nicotinamide riboside particles exhibit less than 20% hydrolysis when incorporated into an aqueous skin care composition.

### FIELD

The present disclosure is directed generally to an encapsulated cosmetic agent. More specifically, the present disclosure is directed to encapsulated nicotinamide riboside particles for use as a skin care agent.

### BACKGROUND

Skin conditions include some of the most common disorders treated in the developing world, and treating such conditions has led to a booming skin care industry that generates billions of dollars in sales each year. Different skin conditions are associated with widely varied triggers, biological mechanisms, environmental factors, and clinical manifestations. For example, as people age, intrinsic factors related to the biochemical changes within the skin typically result in visible signs of skin aging such as wrinkling and other forms of roughness (including increased pore size, flaking and skin lines) and/or uneven skin pigmentation (e.g., age spots or melasma). In some instances, lifestyle choices and exposure to the environment may allow extrinsic factors such as ultraviolet radiation, pollution (e.g., engine exhaust, cigarette smoke, smog), wind, heat, low humidity, harsh surfactants, abrasives, and the like to damage the skin, leading to undesirable skin appearance. As a result, a multitude of cosmetic skin care products have been developed that contain skin care agents tailored to treat common skin conditions.

An example of skin care agents known for use in skin care products are Vitamin B₃ compounds such as niacin and its derivatives. U.S. Pat. No. 4,096,240 refers to niacin as effective in skin lightening. U.S. Pat. No. 8,106,184 discloses treating skin or epithelial cells with a nicotinoyl riboside or derivative compound that increases the level of intracellular nicotinamide adenine dinucleotide NAD+ to treat skin afflictions or skin conditions such as disorders or diseases associated with or caused by inflammation, sun damage or natural aging. U.S. Publication No. 2005/0267023 discloses methods and compositions for modulating the life span of a cell or its resistance to stress, for example, by contacting the cell with nicotinamide riboside to stimulate the NAD+salvage pathway in the cell. PCT Pub. No. WO 2015/066382 (“Deren-Lewis”) relates to methods of using nicotinamide riboside to promote the increase of intracellular levels of (NAD+) in cells and tissues for improving cell and tissue survival. Deren-Lewis discloses the use of topical nicotinamide riboside compositions for treating a variety of skin conditions by modulating the NAD+ pathway.

It has recently been found that nicotinamide riboside (“NR”) may be a suitable skin care agent when applied topically or ingested. But incorporating NR into an aqueous cosmetic composition can be problematic. Many cosmetic compositions include water, and NR tends to hydrolyze in the presence of water. The rate and amount of hydrolysis depends on the amount of water present, the length of time the NR is exposed to the water and the temperature. See, “Kinetic a-Deuterium Isotope Effects for Enzymatic and Nonenzymatic Hydrolysis of Nicotinamide-β-Riboside” by Ferraz, et al., Department of Chemistry, Indiana University, Archives of Biochemistry and Biophysics, Vol. 191, No. 2, pp. 431-436, 1978. Thus, by the time a consumer is ready to use an NR-containing cosmetic product, the NR may be substantially degraded or no longer present. In some instances, it may even be desirable to incorporate NR into ingestible compositions such as beverages, which typically include a substantial amount of water. In these instances, it is particularly important to minimize or prevent hydrolysis of NR in the composition.

US 2012/0015004 (“Mironov”) relates to encapsulated nutrient salts for use in high-acid beverages. However, Mironov does not recognize the skin care benefits that NR can provide, nor that NR hydrolyses when incorporated into an aqueous compositions.

U.S. Pub. Nos. 2003/0207776, 2003/0232091, 2011/10268802, 2011/0269657, and 2015/0099680 disclose examples of encapsulating materials suitable for a wide variety of different uses, but none of these publications recognize the hydrolysis problem encountered when incorporating NR into an aqueous composition or the benefit of encapsulating NR to improve the stability of NR in an aqueous composition.

Accordingly, it would be desirable to inhibit and/or prevent hydrolysis of NR in an aqueous composition by encapsulating the NR with a water insoluble encapsulating agent using a suitable encapsulation technique.

### SUMMARY

The present disclosure provides stable encapsulated skin care agents. In one aspect, the stable encapsulated skin care agent comprises a plurality of encapsulated NR particles. Each particle includes a core surrounded by a shell, wherein the core comprises nicotinamide riboside (NR) and shell comprises an encapsulation agent. In some instances, the encapsulation agent is water impermeable and/or water insoluble, such that the NR contained in the core is not hydrolyzed by any water present in the surrounding environment. In some instances, the encapsulated particles may include multiple shells and/or cores.

The encapsulated particles herein may be made by applying a water insoluble encapsulating agent to a plurality of NR particles using a fluidized bed coater.

### BRIEF DESCRIPTION OF THE DRAWINGS

FIG. 1 is an illustration an encapsulated particle in a single-core, single-shell configuration.

FIG. 2 is an illustration of an encapsulated particle in a single-core, multiple-shell configuration.

FIG. 3 is an illustration of an encapsulated particle in a multiple-core, single-shell configuration.

### DETAILED DESCRIPTION

The susceptibility of NR to hydrolysis in an aqueous solution limits its usefulness in skin care compositions, many of which tend to be aqueous. In order to reduce and/or prevent the hydrolysis of NR in an aqueous composition, it has now been found that coating NR with an encapsulation agent improves the stability of the NR in aqueous compositions.

Materials, features, structures and/or characteristics of the encapsulated skin care agent described herein may be combined in any suitable manner across different embodiments, and materials, features, structures and/or characteristics may be omitted or substituted from what is described. Thus, embodiments and instances described herein may comprise or be combinable with elements or components of other embodiments and/or instances despite not being expressly exemplified in combination, unless otherwise stated or an incompatibility is stated.

All percentages are by weight of the cosmetic composition or encapsulated particles, as indicated, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The cosmetic compositions herein can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions.

“About” means

“Apply” or “application”, as used in reference to a composition, means to apply or spread the compositions of the present invention onto a human skin surface such as the epidermis.

“Aqueous composition” refers to a composition that contains at least 20% water.

“Cosmetic” means providing a desired visual effect on an area of the human body. The visual cosmetic effect may be temporary, semi-permanent, or permanent.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (“GRAS”) by the U.S. Food and Drug Administration, food additives, and materials used in non-cosmetic consumer products including over-the-counter medications. The compositions herein may optionally include one or more cosmetic agents in addition to nicotinamide riboside. In some embodiments, cosmetic agents may be incorporated in a cosmetic composition comprising a dermatologically acceptable carrier suitable for topical application to skin.

“Effective amount” means the amount of encapsulated nicotinamide riboside sufficient to provide the desired skin benefit (e.g., improve the appearance of a hyperpigmented spot) over the course of a treatment period.

“Encapsulated” means that at least 80% of the surface area of a nicotinamide riboside particle is covered by an encapsulating agent. For example, at least 85%, 90%, 91%, 92%, 93%, 94%, 95%, 96%, 97, 98%, 99%, and ideally 100% of the surface area of an encapsulated NR particle is covered by an encapsulating agent.

“Water impermeable” refers to a material through which water and other fluids cannot pass without catastrophic failure of the material (e.g., rupturing, tearing, breaking, melting, or dissolving).

“Generally recognized as safe” or “GRAS” refers to a material that complies with Sections 201(s) and 409 of the Federal Food, Drug, and Cosmetic Act, and the U.S. Food and Drug Administration's implementing regulations in 21 CFR 170.3 and 21 CFR 170.30, which require the premarket review and approval by the FDA of any use of a food substance, unless the substance is generally recognized, among qualified experts, as having been adequately shown to be safe under the conditions of its intended use either through scientific procedures or, for a substance used in food before 1958, through experience based on common use in food.

“Skin care agent” means a cosmetic agent for regulating and/or improving a skin condition. Some nonlimiting examples of regulating and/or improving a skin condition include improving skin appearance and/or feel by providing a smoother, more even appearance and/or feel; increasing the thickness of one or more layers of the skin; improving the elasticity or resiliency of the skin; improving the firmness of the skin; reducing the oily, shiny, and/or dull appearance of skin; improving the hydration status or moisturization of the skin; improving the appearance of fine lines and/or wrinkles; improving skin exfoliation or desquamation; plumping the skin; improving skin barrier properties; improving skin tone; reducing the appearance of spots, redness or skin blotches; and/or improving the brightness, radiancy, or translucency of skin. Skin care agents may incorporated in topical compositions for directed application to a target skin area, or incorporated into an ingestible composition such as a beverage and delivered to a target skin portion via the digestive and circulatory systems of the body.

“Stable” means that a composition or ingredient retains a desired level of potency for the duration of predetermined expiration period, as defined by generally accepted pharmaceutical or cosmetological protocols (e.g., good manufacturing practices (“GMP”), or as promulgated by various trade conventions such as, for example, the United States Pharmacoepia Convention. For example, a stable, encapsulated NR particle may exhibit less than 20% hydrolysis of the NR contained in the core of the particle (e.g., less than 15%, 10%, 5%, 4%, 3%, 2%, or even less than 1%) when placed in an aqueous solution at between 15 and 40° C.±2° C. (e.g., 16° C., 18° C., 20° C., 22° C., 24° C., 26° C., 28° C., 30° C., 32° C., 34° C., 36° C., 38° C., 40° C.,±2° C.) for at least 1 hour (e.g., at least 2 hours, 5 hours, 8 hours, 12 hours, or even at least 24 hours). In some instances, the encapsulated skin care agent herein may be stable in an aqueous composition at 15-40° C. for more than 1 day, 2 days, 3 days, 4 days, 5 days, 6 days, 7 days or even for more than 2 weeks, 1 month, 2 months, 3 months, 4 months, 5 months, or even more than 6 months. Stability may be determined according to the Hydrolysis Test described in more detail below.

“Treatment period,” as used herein means the length of time and/or frequency that the encapsulated skin care agent is used. The treatment period may be a predetermined length of time and/or frequency, but need not necessarily be so.

“Water insoluble” refers to a material that does not readily dissolve in water (e.g., has a water solubility at 25-50° C. of less than 200 millimoles/liter, less than 100 millimoles/liter, less than 50 millimoles/liter or even less than 10 millimoles/liter).

### Encapsulated Cosmetic Agent

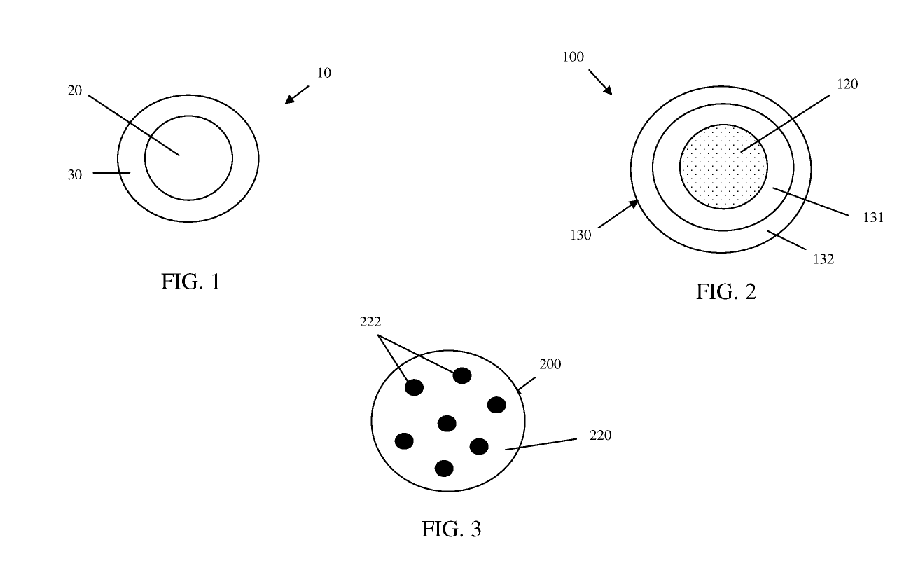

The encapsulated cosmetic agent herein is a stable NR-containing particle. The stable NR-containing particle includes NR, and any optional additional ingredients, coated with an encapsulating agent to provide encapsulated particles in a core-shell configuration, for example, as illustrated in FIGS. 1, 2 and 3, which are described in more detail below. The shell may be 5% to 80% (e.g., 10% to 40%) of the weight of the particle and have a total shell thickness of 10 nanometers (“nm”) to 1 millimeter (“mm”) (e.g., between 10 nm and 500 micrometers (“μm”), 20 nm and 300 μm, 50 nm and 200 μm, 100 nm, and 100 μm, 200 nm and 1 μm, 300 nm and 500 nm, or even between 300 nm and 400 nm). The core may include 1% to 99% NR based on the weight of the particle. The encapsulated particles herein generally have a weight average particle size of less than 500 microns (μm) (e.g., less than 400 μm, 300 μm, 250 μm, 200 μm, 150 μm, 100 μm or even less than 50 μm) but typically larger than 1 μm (e.g., larger than 10 μm, 20 μm, 50 μm or even larger than 100 μm). Particles larger than 500 μm, or even larger than 300 μm, may be unsuitable for use in certain compositions such as beverages because they tend to import an undesirable gritty texture to the beverage. In some instances, particles larger than 100 μm may not he suitable for use in topical compositions because they can be harder to suspend in a cosmetic composition leading to a gritty and/or non-homogenous feel. On the other hand, particle sizes less than 1 μm have a surface area-to-volume ratio that undesirably favors increased hydration of the core relative to larger particles. And smaller particles may introduce undesirable processing difficulties and/or safety concerns.

In some instances, it may be desirable to communicate to a user that the NR is present in a composition containing the present particles, such as a beverage, by making the encapsulated particles visible. Visible particles may be provided by any suitable method known for imparting visibility to particles in a beverage, for example, by including GRAS pigments and/or dyes.

FIG. 1 is an illustration of an encapsulated particle 10that includes a solid spherical core 20 surrounded by an encapsulating shell 30. The shell 30 includes an encapsulating agent and provides a water barrier between the core 20 and the external environment, which prevents or at least inhibits water from hydrolyzing NR in the core and/or in an underlying layer. The barrier may he dissolvable, water insoluble, and/or water impermeable. It may be desirable for the shell 30to completely surround the core 30, as shown in FIG. 1, in order to adequately insulate the water-sensitive NR in the core 20 from contact with any water present in the external environment. But it is to he appreciated that some of the particles herein may have less than 100% of the core covered by the encapsulating agent. It is also to be appreciated that the core 20 can be any shape, as desired.

In some instances, the encapsulated particle may include a shell comprising more than one layer of the same or different materials. For example, the encapsulated particle may comprise a multi-layer shell in which a first, outer layer functions as a water insoluble barrier and a second, inner layer functions to scavenge any water that penetrates the first layer, thereby reducing the amount of water available to hydrolyze the NR in the particle. In some instances, the encapsulated particle may include multiple dissolvable barrier layers and/or NR-containing layers, for example, to provide an encapsulated particle that releases a desired amount of NR over a predetermined period of time (“controlled release particle”). The controlled release particle in this example may include alternating barrier layers and NR-containing layers.

FIG. 2 is an illustration of an encapsulated particle 100 with a multi-layer shell 130 surrounding an NR-containing core 120. The NR in the core 120 may be solid (i.e., contains less than 5% liquid), dissolved in a miscible fluid or dispersed in an immiscible fluid. The multi-layer shell 130 includes a first, outer layer 132 and a second, inner layer 131. While not shown in FIG. 2, it is to be appreciated that the encapsulated particle 100 may, optionally, include one or more additional layers disposed around the first layer 132 and/or second layer 131. The first layer 132, second layer 131, and optional additional layers may be made from the same or different material and may provide the same or different functions, as desired. Each layer 131, and 132 of the multi-layer shell 130 may have the same or different thickness (e.g., between 1 nm and 500 μm, 10 nm and 300 μm, 50 nm and 100 μm, 100 nm and 50 μm, or even between 200 nm and 1 μm), as long as the NR in the encapsulated particle 100 is able to provide the desired skin care benefit.

In some instances, the encapsulated particles herein may include multiple cores surrounded by a continuous, unitary shell, for example, as illustrated in FIG. 3. FIG. 3 shows an encapsulated particle 200 that includes multiple NR-containing cores 222 surrounded by a continuous shell 220. Multi-core encapsulated particles like the one illustrated in FIG. 3 may be made using known processing techniques such as prilling, spray chilling, spray drying microfluidics, extrusion and loading a porous carrier.

### Core

The encapsulated cosmetic agent herein includes a nicotinamide riboside containing core coated with an encapsulating agent. Nicotinamide riboside (CAS No. 1341-23-7) has the formula:

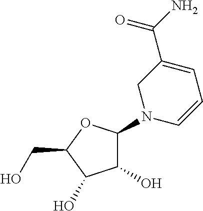 [SMILES: NC(=O)C1=CC=CN([C@@H]2O[C@H](CO)[C@@H](O)[C@H]2O)C1]

Some examples of nicotinamide riboside and its methods of manufacture are described in U.S. Pat. No. 8,106,184. As used herein, the term “nicotinamide riboside” includes salts of nicotinamide riboside (e.g., nicotinamide riboside chloride). Nicotinamide riboside may be obtained from ChromaDex, Inc., Irvine, Calif. The encapsulated particles herein contain at least 1% NR, based on the weight of the particle, but typically less than 90% (e.g., from 5% to 90%, 20% to 70% or even from 40% to 60%).

In addition to NR, the core may optionally include one or more other ingredients commonly included in cosmetic compositions (e.g., colorants, skin tone agents, skin anti-aging agents, nutritional supplements such as vitamins and minerals, anti-inflammatory agents, sunscreen agents, combinations of these and the like), provided that the additional ingredients do not undesirably alter the skin care benefit provided by the NR. The additional ingredients should be suitable for use in contact with human skin tissue without undue toxicity, incompatibility, instability, allergic response, and the like. Some nonlimiting examples of additional ingredients which may be suitable for use herein are described in U.S. Publication Nos. 2002/0022040; 2003/0049212; 2004/0175347; 2006/0275237; 2007/0196344; 2008/0181956; 2010/00092408; 2008/0206373; 2010/0239510; 2010/0189669; 2011/0262025; 2011/0097286; US 2012/0015004 US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

### Shell

The encapsulating agent(s) herein form a barrier around the NR-containing core to provide a suitable barrier between the NR-containing core and the external environment. The shell formed by the encapsulating agent may be frangible or pliable (e.g., a plastic, elastic, or plastoelastic film), as long as the NR in the particle is released as intended. It may be desirable to provide an encapsulating shell that releases the NR when the particle is subjected to the shearing and/or crushing force typically experienced during topical application of a cosmetic composition. Additionally or alternatively, it may be desirable to provide an encapsulating shell that releases the NR when the particle is exposed to one or more conditions typically found in the gastrointestinal tract of a human. Encapsulating agents that may be used herein are not particularly limited and can include any suitable GRAS material that provides a desirable combination of barrier and NR release properties. Some non-limiting examples of encapsulating agents that may be suitable for use herein are chitin and chitosan; cellulose and cellulose derivatives such as cellulose acetate phthalate, hydroxypropyl methyl cellulose, carboxymethyl cellulose, enteric/aquateric coatings and mixtures thereof; silicates, phosphates, and borates polyvinyl alcohol; polyvinyl acetate/polyvinyl alcohol blends; polyethylene glycols; linear and branched carbohydrates such as simple sugars onosaccharides) and mixtures thereof, oligosaccharides (2-10 monosaccharide units), and polysaccharides (35 or greater monosaccharide units) and mixtures of these; carbohydrates that have been modified to improve their water resistance properties (e.g., by adding alkyl or aryl functionalities); waxes; oil-in-water emulsions comprising silicone oils, silicone gels, or silicone elastomers suspended in water; aqueous latex dispersions comprising film forming polymer particles of polyacrylate, polyurethanes, silicas, and silicones, which upon dehydration coalesce to make uniform, low permeability films. The encapsulating agents may optionally include plasticizers such as, for example, sorbitol, polyethylene glycol and polypropylene glycol to help achieve a more homogeneous, impermeable coating. Plasticizers when included in the encapsulating agent may be present at from 0.01% to 10% by weight, based on the weight of particle.

In some instances, the shell may be in the form a discrete, continuous layer of material that surrounds the core, for example, as illustrated in FIGS. 1 and 2. In some instances, the shell may be in form of a solid matrix in which particles of NR (solid or contained in a liquid) are dispersed or suspended, for example, as illustrated in FIG. 3.

Methods of Making

The stable skin care agent herein may be made using conventional methods of encapsulating a water soluble active to provide a stable particle for use in an aqueous composition. In particular, the encapsulated skin care agent herein may be made by applying one or more coatings of an encapsulating agent to an NR-containing material. It may be desirable to use an encapsulating agent and/or process that hydrolyzes less than 20% of the NR in the core is hydrolyzed during encapsulation. Some non-limiting examples of methods of providing the encapsulated particles herein are coacervation, polycondensation, interfacial polymerization, emulsion polymerization, solvent evaporation, solvent exchange, lyophilization, nanoprecipitation, spray drying, spray chilling, prilling, extrusion, and fluid bed coating. Additional non-limiting examples of particle formation, encapsulation and/or coating techniques are disclosed in U.S. Pat. Nos. 5,550,119; 7,338,928; 6,790,821; 8,236,715; and 8,945,419; 9,029,083; 9,039,273 and U.S. Publication Nos. 2003/0207776; 2003/0232091; 2004/0096515; 2005/0276831; 2006/0078893; 2007/0054119; 2007/0092914; 2009/0197772; 2010/0104712; 2010/0158984; 20100159079; 2010/0213628; 2011/10268802; 2011/0269657; 2011/0143985; 2011/0143984; 2012/0015004; 2012/0077880; 2012/0077881; 2014/0065234; 2014/0220087; 2014/0178964; 2015/0099680; and 2015/0010600.

In some instances, NR powder particles can be directly coated with an encapsulation agent using a fluidized bed coating/drying operation, which results in particles with a solid core. For example, a Wurster brand fluidized bed coater or equivalent may be used to provide a continuous, unbroken coating around NR powder particles. In this example, the NR powder is sprayed with a suitable coating material (e.g., an aqueous solution of film forming polymers or a meltable, hydrophobic material that solidifies or crystallizes on the surface of the NR core). The spray-on encapsulation agent may be in the form of a suspension, emulsion or dispersion. The fluidized bed is operated such that the flux number of the fluid bed is between 3.5 and 7 (e.g., between 3.5 and 5.0) and the Stokes number is greater than 1 (e.g., between 1.0 and 1000 or between 100 and 1000). The flux number provides and estimation of the operating parameters of a fluidized bed to control coating within the bed, and the Stokes number is a measure of particle coalescence for describing the degree of mixing occurring to particles in the fluid bed. U.S. Pat. No. 6,790,821 to Wasserman, et al., describes how to determine flux number and Stokes number. The sprayed particles in the fluidized bed are then dried with dehumidified air maintained below the degradation temperature of the NR. The resulting coated particles may have a weight average particle size of between 20 and 800 microns.

Optionally, the NR may be mixed with inert materials and binders prior to the fluidized bed process to achieve a particle size that is appropriate for fluidization, For example, particles <20 micrometers are typically not appropriate for fluidization, as they tend to elutriate out of the bed), and particles greater than 800 microns are not appropriate for fluidization, as they tend to require undesirably high fluidization velocities.

The fluid bed mixer includes at least one coating zone where the encapsulation agent is applied. The coating zone involves the spraying of the encapsulation agent onto the fluidized particles. The bed may be fluidized with heated air. Spraying may be achieved via nozzles capable of delivering a fine or atomized spray of the encapsulation agent to achieve complete coverage of the particles. Typically, the droplet size from the atomizer is less than 2 times the particle size. This atomization can be achieved either through a conventional two-fluid nozzle with atomizing air, or alternatively by means of a conventional pressure nozzle. It may be desirable to position the nozzle above the fluidized height of the particles in the fluid bed to allow a vertical down spray of the coating mixture (i.e., a top spray configuration). The coating zone of the fluid bed may be followed by a drying zone and a cooling zone. It is to be appreciated that alternative arrangements are also possible to achieve the desired coated particles.

Typical conditions within a fluid bed apparatus include: (i) from 1 to :20 minutes of mean residence time; (ii) from 100 to 600 mm of depth of unfluidized bed; (iii) a droplet size of less than 2 times the size of the particles, (e.g., not more than 100 μm or 50 microns); (iv) from 150 to 1600 mm of spray height from the fluid bed plate or preferably 0-600 mm from the top of the fluid bed, (v) from 0.1 to 4.0 m/s of fluidizing velocity, preferably 1.0 to 3.0 m/s; and (vi) from 12 to 200° C. of bed temperature (e.g., 15 to 100° C.). Again, one of ordinary skill in the art will recognize that the conditions in the fluid bed may vary depending on a variety of known factors.

In some instances, NR powder may be dissolved in a miscible solvent, and droplets of the resulting NR-containing solution can be encapsulated using known chemical or physical encapsulation techniques, resulting in the formation of encapsulated particles with a liquid core. Some non-limiting examples of solvents that can dissolve NR are 3-methyl isoxazole, acetanilide, succinic anhydride, pyridazine, 1-methyl imidazole, salicylaldehyde, tetrahydrofurfuryl alcohol, 2-pyrolidone, 2-pyrrolidinone, isoxazole, dimethyl sulfone, tetramethylene sulfone, thiazole, thiourea, b-propiolactone, ethylene cyanohythin, dimethyl sulfoxide, dimethyl sulfoxide, 1,3 -triazole, diethylenetriamine, diethylenetriamine, dimethyl formamide, n,n-dimethylformamide, 2-chloropropenoic acid, acetonecyanhydrin, shellac, polyethylene oxide 4000, sorbitol and mixtures of these.

In some instances, NR powder can be dispersed in an immiscible solvent, and the dispersion can then be encapsulated using chemical or physical encapsulation techniques known in the art for encapsulation of lipophilic oils, resulting in the formation of encapsulated particles with a liquid core. Some non-limiting examples of immiscible solvents are mono, di- and tri-esters of C4-C24 fatty acids and glycerin; fatty acid esters of polyglycerol oligomers polyalphaolefins, butyl oleate, hydrogenated castor oil, sucrose benzoate, dodecanoic acid, palmitic acid, stearic acid, octadecanoic acid, monoester with 1,2,3-propanetriol; dodecanoic acid, pentyl ester; octanoic acid, nonyl ester; pentadecanoic acid, ethyl ester; hexadecanoic acid, methyl ester; dodecanoic acid, 4-methylphenyl ester; dodecanoic acid, 3-methylbutyl ester; tetradecanoic acid, 1-methylethyl ester; hexadecanoic acid; 1-phenanthrenecarboxylic acid, hexarose; butyl oleate; hydrogenated castor oil; isopropyl myristate; castor oil: mineral oil; isoparaffin; capryllic triglyceride; soybean oil; vegetable oil; geranyl palmitate; silicones; polydimethylsiloxane; heptadecane; isododecane; perfume raw materials with a Calculated logP (“ClogP”) of greater than 5 using the CLOGP program available from Daylight Chemical Information Systems Inc., Irvine, Calif.

In some instances, an NR-solution (e.g., NR dissolved in a miscible liquid) may be dispersed in a meltable immiscible solvent, which is then prilled or spray chilled to produce encapsulated particles. In a prilling operation, the melted suspension is dosed onto a centrifugal atomizer. The centrifugal atomizer generates atomized particles that are subsequently cooled in the air.

In some instances, solubilized NR can be pre-loaded into a porous carrier such as zeolites, precipitated silicas or lattice-network microspheres, and then encapsulated according to one of the aforementioned encapsulation techniques. In this way, the solubilized NR contained in the pores of the porous carrier is protected from hydrolysis by the lattice structure of the carrier and the encapsulation agent, which work cooperatively o hinder hydrolysis of the NR.

In some instances, the NR powder (either dissolved in a miscible solvent or dispersed in an immiscible carrier fluid) can be encapsulated in single or multiple shells using a microfluidic technique. To form a single shell, the NR-containing fluid and shell materials/precursors are pushed through a concentric nozzle, then emulsified into drop-in-a-drop (double emulsion) by a continuous phase fluid. By adding extra flow channels, additional shell layers can be formed to yield a microcapsule suspension at provides adequate hydrolysis stability to NR when dosed in a finished product formulation. U.S. Publication No. 2008/0213593 discloses microlluidic techniques that may be suitable for use herein.

In some instances, the encapsulated particles herein may be coated with a material to reduce the rate of leakage of NR from the particles when the particles are subjected to a bulk environment (e.g., storage and shipping). Some non-limiting examples of such materials include polyvinyl pyrrolidone homopolymer, and its various copolymers with styrene, vinyl acetate, imidazole, primary and secondary amine containing monomers, methyl acrylate, polyvinyl acetal, maleic anhydride; polyvinyl alcohol homopolymer, and its various copolymers with vinyl acetate, 2-acrylamide-2-methylpropane sulfonate, primary and secondary amine containing monomers, imidazoles, methyl acrylate; polyacrylamides; polyacrylic acids; microcrystalline waxes; paraffin waxes; modified polysaccharides such as waxy maize or dent corn starch, octenyl succinated starches, derivatized starches such as hydroxyethylated or hydroxypropylated starches, carrageenan, guar gum, pectin, xanthan gum; modified celluloses such as hydrolyzed cellulose acetate, hydroxy propyl cellulose, methyl cellulose, and the like; modified proteins such as gelatin; hydrogenated and non-hydrogenated polyalkenes; fatty acids; hardened shells such as urea crosslinked with formaldehyde, gelatinpolyphosphate, melamine-formaldehyde, polyvinyl alcohol crosslinked with sodium tetraborate or gluteraldehyde; latexes of styrene-butadiene, ethyl cellulose; and mixtures thereof.

The encapsulated NR particles herein may be used anywhere stable NR particles are desired. For example, the encapsulated NR particles may be incorporated into a cosmetic composition such as a skin care composition or a beverage to improve the appearance of skin or to treat a skin condition.

### Hydrolysis Test

The Hydrolysis Test provides a method for determining the stability of NR. In particular, this method can be used to determine the amount of NR that is hydrolyzed when incorporated into an aqueous composition.

Sample Preparation

If the NR particles are not in a suitable aqueous vehicle (i.e., a skin care product or other composition that simulates an aqueous skin care product), mix the NR particles into a suitable aqueous vehicle. Table 1 below is provided as an example of ingredients that can be combined to form a suitable aqueous vehicle. The NR particles may be added at any amount desired, but are typically included at from 0.1% to 5% w/v. The ingredients may be combined using conventional methods of making skin care compositions. After the skin care composition is made, weigh 0.1 g of the skin care composition into a polypropylene conical centrifuge tube, and dilute with 25 mL of a diluent. The diluent is made from 5% (v/v) 5 mM ammonium formate, 0.025% (v/v) formic acid in Milli-Q water and 95% (v/v) acetonitrile. Vortex or homogenize as needed to disperse the product formulation in the diluent. Using a syringe, filter a sufficient amount of the sample into an autosampler vial for HPLC analysis as described below. Prepare standard stock solutions in Milli-Q water for calibration. Dilutions are made into diluent to cover a range of approximately 5-350 μg/mL analyte in solution for calibration curves.

TABLE 1

| Component | % |
|---|---|
| Phase A | |
| water | qs |
| glycerol | 3.00 |
| disodium EDTA | 0.10 |
| Phase B | |
| Isopropyl Isostearate | 1.33 |
| Isohexadecane | 3.00 |
| cetearyl glucoside | 0.20 |
| cetyl alcohol | 0.32 |
| tocopherol acetate | 0.50 |
| PEG-100 stearate | 0.10 |
| stearyl alcohol | 0.48 |
| behenyl alcohol | 0.40 |
| ethyl paraben | 0.20 |
| propyl paraben | 0.10 |
| polymethylsilsesquioxane | 0.25 |
| Phase C | |
| polyacrylamide/C13-14 | 2.00 |
| isoparaffin/laureth-7 | |
| Phase D | |
| benzyl alcohol | 0.25 |
| dimethicone/dimethiconol | 2.00 |

### HPLC

An Alliance 2695 brand HPLC system with 996 PDA detector (Waters, Milford, Mass.) or equivalent with the separation mode set to hydrophilic interaction chromatography (HILIC) is used as the chromatographic system. Inject 5 microliters of the diluted formulation samples or calibration standards into the column. Nicotinamide riboside is separated from other components in the product on a SeQuant ZIC-Hilic (4.6×100 mm; 5 micron particle size) stationary phase. Hold the column temperature at 30° C. The mobile phase is: (A) 5 mM ammonium formate with 0.025% (v/v) formic acid in Milli-Q water; and (B) a mixture of 95% acetonitrile and 5% mobile phase (A) also with 0.025% (v/v) formic acid. Begin the gradient at 100% (B) and hold for 3 minutes. Next, use 60% (B) until 16 minutes, and hold for 3 minutes before returning to the starting condition of 100% (B). The entire chromatographic run should take about 24 minutes using a flow rate of 1.0 mL/min Table 2 shows the times and gradients used in the test. The diode array detector is set to scan wavelengths of 205-350 nm Chromatograms are extracted at 260 nm Retention time is approximately 12.6 minutes. Quantitation is performed using Chromeleon v.7.2 or equivalent chromatography data system software package. A linear curve fit of the response of the calibration standards is used to determine analyte in solution levels. Results are expressed as weight percent (w/w %) once corrected for the dilution factor and weight of formulation aliquot.

TABLE 2

| Time (min) | % A | % B |
|---|---|---|
| 0 | 0 | 100 |
| 3 | 0 | 100 |
| 16 | 40 | 60 |
| 19 | 40 | 60 |
| 24 | 0 | 100 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable skin care agent, comprising: a plurality of encapsulated particles in a core-shell configuration, wherein the core comprises nicotinamide riboside (NR) and the shell comprises an encapsulating agent.

2. The stable skin care agent of claim 1, wherein less than 20% of the encapsulated nicotinamide riboside is hydrolyzed according to the Hydrolysis Test.

3. The stable skin care agent of claim 1, wherein the shell is at least one of water impermeable and water insoluble.

4. The stable skin care agent of claim 1, wherein the NR is nicotinamide riboside chloride.

5. The stable skin care agent of claim 1, further comprising a weight average particle size of between about 1 and about 500 microns.

6. The stable skin care agent of claim 1, wherein the core is a solid core.

7. The stable skin care agent of claim 1, wherein the core is a liquid or semi-solid.

8. The stable skin care agent of claim 7, wherein the core is a liquid core and the NR is dissolved in a miscible solvent.

9. The stable skin care agent of claim 8, wherein the miscible solvent is sorbitol, propylene glycol or a mixture of these.

10. The stable skin care agent of claim 7, wherein the core is a liquid core and the NR is dispersed in an immiscible fluid.

11. The stable skin care agent of claim 10, wherein the immiscible fluid is glycerin.

12. The stable skin care agent of claim 1, wherein the core comprises a porous carrier with the NR disposed thereon.

13. The stable skin care agent of claim 12, wherein the porous carrier is selected from zeolites, precipitated silicates, microspheres and combinations thereof.

14. The stable skin care agent of claim 1, wherein the encapsulated particles each comprise two or more cores surrounded by a unitary shell.

15. The stable skin care agent of claim 1, wherein the encapsulated particles each comprise one core surrounded by two or more shells.

16. The stable skin care agent of claim 1, wherein the core and the shell include only materials that are generally recognized as safe (GRAS).

17. A method of making the stable skin care agent of claim 1, comprising applying a water insoluble encapsulating agent to a plurality of NR particles using a fluidized bed coater.
